(19) United States
(12) Patent Application Publication
(10) Pub. No.: US 2024/0302253 A1
(43) Pub. Date: Sep. 12, 2024

(54) SYSTEMS AND METHODS FOR TISSUE CUTTING SYSTEM WITH VISION

(71) Applicant: Elephas Biosciences Corporation, Madison, WI (US)

(72) Inventors: Christian Baltes, Madison, WI (US); Todd Bakken, Madison, WI (US); Wesley Cox-Muranami, Madison, WI (US); Jonathan Daniel Oliner, Garrett Park, MD (US); Ryan Fischer, Madison, WI (US)

(21) Appl. No.: 18/599,465

(22) Filed: Mar. 8, 2024

Related U.S. Application Data
(60) Provisional application No. 63/450,892, filed on Mar. 8, 2023.

(51) Int. Cl. G01N 1/28; G01N 33/483

(52) U.S. Cl. CPC G01N 1/286; G01N 33/4833; G01N 2001/2873

(57) ABSTRACT

The present invention relates to devices, systems, and methods for cutting tissues. In some embodiments, the devices, systems, and methods of the invention relate to cutting tissues into fragments that find use in tissue culture and drug testing applications.

### CROSS-REFERENCE TO RELATED APPLICATIONS

This application claims the benefit of priority to U.S. Provisional Patent Application No. 63/450,892, filed Mar. 8, 2023, which is hereby incorporated by reference in its entirety.

### FIELD

The present invention relates to devices, systems, and methods for cutting tissues. In some embodiments, the devices, systems, and methods of the invention relate to cutting tissues into fragments that find use in tissue culture and drug testing applications.

### BACKGROUND

Various diagnostic applications require tissue to be cut into thin sections. Conventional methods are limited by the speed of cutting and require one or more manual steps. As such, an unmet need exists to cut tissue into sections at high speed and precision, in an automated manner without causing significant mechanical damage to the tissue. It is also desirable to maintain maximum tissue viability for various downstream applications requiring live tissue, such as ex vivo drug response determination in various precision oncology applications.

### SUMMARY

One aspect of the present disclosure provides a tissue cutting system comprising: a tissue holder configured to hold a tissue sample. The tissue holder includes an opening configured to expose a portion of the tissue sample. The tissue cutting system further includes a reservoir, and a portion of the tissue holder is positioned within the reservoir. The tissue cutting system further includes a cutting component configured to cut the tissue sample contained within the tissue holder; and a camera configured to image the opening of the tissue holder and the portion of the tissue sample.

In some embodiments, the tissue holder is movable between a first position for cutting by the cutting component and a second position for imaging by the camera.

In some embodiments, the tissue cutting system further includes a translation assembly coupled to the tissue holder and configured to move the tissue holder containing the tissue sample with respect to the cutting component, the camera, or a combination thereof.

In some embodiments, the translation assembly includes a translation stage, the reservoir and the tissue holder are mounted to the translation stage.

In some embodiments, the translation stage is configured to move in two degrees of freedom in a translation plane.

In some embodiments, the camera defines a camera axis that intersects the translation plane.

In some embodiments, the tissue cutting system further includes a mirror positioned in the translation plane.

In some embodiments, the camera defines a camera axis that is perpendicular to a longitudinal axis of the tissue holder.

In some embodiments, the tissue cutting system further includes a light source configured to illuminate the opening of the tissue holder.

In some embodiments, the light source is coupled to the reservoir or a cover on the reservoir.

In some embodiments, the light source defines an illumination axis that intersects a longitudinal axis of the tissue holder.

In some embodiments, the light source is intermittently energized according to when the camera captures an image of the opening of the tissue holder.

In some embodiments, the light source is energized to emit a first light with a first spectral distribution and is also energized to emit a second light with a second spectral distribution.

In some embodiments, the tissue cutting system further includes a light pipe configured to direct a light emitted from the light source to the opening of the tissue holder.

In some embodiments, the light pipe is formed in the reservoir.

In some embodiments, the light source is positioned at least partially within the reservoir.

In some embodiments, the tissue cutting system further includes a filter wheel coupled to the camera.

In some embodiments, the reservoir is filled with a fluid material, wherein the portion of the tissue holder is submerged within the fluid material.

In some embodiments, the reservoir includes an optical window positioned opposite the tissue holder.

In some embodiments, the tissue cutting system further includes a drive assembly configured to move the tissue sample relative to the tissue holder along a longitudinal axis of the tissue holder, and a rotating device coupled to the tissue holder and is configured to rotate the tissue holder about the longitudinal axis.

In some embodiments, the cutting component is a first cutting component configured to cut a tissue sample contained within the tissue holder by scoring cuts in a first dimension and a second dimension, and wherein the tissue cutting system further includes a second cutting component configured to cut the tissue sample in a third dimension to produce tissue fragments.

In some embodiments, the camera images the tissue sample after the cutting component has cut the tissue sample.

In some embodiments, the tissue sample is a live tissue sample.

Another aspect of the present disclosure provides a method comprising: capturing a first image of a first cross-section of a sample; identifying a first target area in the first image; determining a first set of cutting parameters based on the first target area; cutting the sample with the first set of cutting parameters; capturing a second image of a second cross-section of the sample; identifying a second target area in the second image; determining a second set of cutting parameters based on the second target area; and cutting the sample with the second set of cutting parameters.

In some embodiments, the method further includes determining a target volume based on the first image and the second image.

In some embodiments, the first set of cutting parameters includes a variable sized scoring pattern.

In some embodiments, cutting the sample creates a plurality of target tissue fragments of a first size and a plurality of waste fragments of a second size.

In some embodiments, the second size is larger than the first size.

Another aspect of the present disclosure provides a method comprising: moving a sample to a first distance from a camera; capturing a first image of the sample with the camera; determining a first focus value of the first image; moving the sample to a second distance from the camera; capturing a second image of the sample with the camera; determining a second focus value of the second image; and determining a focused distance from the camera based on the first focus value and the second focus value.

In some embodiments, the method further includes capturing a focused image of the sample with the camera with the sample positioned the focused distance from the camera.

In some embodiments, the method further includes measuring a surface area of the sample based on the focused image.

In some embodiments, the method further includes determining the sample is loaded improperly when the first focus value and the second focus value are below a threshold.

Other aspects of the disclosure will become apparent by consideration of the detailed description and accompanying drawings.

### BRIEF DESCRIPTION OF THE DRAWINGS

These and other features, aspects, and advantages of the present technology will become better understood with regards to the following drawings. The accompanying figures and examples are provided by way of illustration and not by way of limitation.

FIG. 1 is a perspective view of a tissue cutting system with a camera.

FIG. 2 is a cross-sectional view of the tissue cutting system of FIG. 1, illustrating the camera.

FIG. 3 is a perspective view of a reservoir of the tissue cutting system of FIG. 1.

FIG. 4 is a cross-sectional view of the reservoir of FIG. 3.

FIG. 5 is a cross-sectional view of a tissue cutting system, illustrating a light source illuminating a tissue holder.

FIG. 6 is an image of a tissue sample positioned within a tissue holder as captured by the camera of FIG. 2.

FIG. 7 is a flowchart illustrating a method of determining a focused distance for a camera and a sample.

FIG. 8 is a flowchart illustrating a method of determining a focused distance for a camera and a sample.

FIGS. 9A-9C are schematics of various tissue sample cross-sections positioned within a tissue holder, illustrating a unique target area for each cross-section.

FIGS. 10A-10C are images of various tissue sample cross-sections positioned within a tissue holder, illustrating a unique target area for each cross-section.

FIG. 11 is a flowchart of a method for dynamically adjusting cutting parameters as a tissue sample is cut.

FIG. 12 is a photo of a front view of a tissue cutting system.

FIG. 13 is a photo of a rear view of the tissue cutting system of FIG. 12.

FIG. 14 is a photo of a reservoir illuminated by a light source of the tissue cutting system of FIG. 12.

FIG. 15 is another photo of the reservoir and light source of FIG. 14.

FIG. 16 is another photo of the reservoir and light source of FIG. 14.

FIG. 17 is a schematic illustrating location targeting of a biopsy, for example, with the tissue cutting system.

FIG. 18 is a schematic illustrating rotational targeting of a biopsy, for example, with the tissue cutting system.

FIG. 19 is a schematic illustrating rotational target with two vertical cuts of a biopsy, for example, with the tissue cutting system.

Before any embodiments are explained in detail, it is to be understood that the invention is not limited in its application to the details of construction and the arrangement of components set forth in the following description or illustrated in the following drawings. The invention is capable of other embodiments and of being practiced or of being carried out in various ways.

### DETAILED DESCRIPTION

The singular forms “a” “an” and “the” include plural referents unless the context clearly dictates otherwise. Approximating language, as used herein throughout the specification and claims, may be applied to modify any quantitative representation that could permissibly vary without resulting in a change in the basic function to which it is related. Accordingly, a value modified by a term such as “about” is not to be limited to the precise value specified. Unless otherwise indicated, all numbers expressing quantities of ingredients, properties such as molecular weight, reaction conditions, so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least each numerical parameter should at least be construed in light of the number of reported significant digits and by applying ordinary rounding techniques.

The phrase “and/or,” as used herein in the specification and in the claims, should be understood to mean “either or both” of the elements so conjoined, e.g., elements that are conjunctively present in some cases and disjunctively present in other cases.

Use of ordinal terms such as “first,” “second,” “third,” etc., in the claims or specification to modify an element does not by itself connote any priority, precedence, or order of one claim element over another or the temporal order in which acts of a method are performed but are used merely as labels to distinguish one element having a certain name from another element having the same name.

The term “coupled,” as used herein, is defined as “connected,” although not necessarily directly, and not necessarily mechanically. The term coupled is to be understood to mean physically, magnetically, chemically, electrically, fluidly or otherwise coupled, connected or linked and does not exclude the presence of intermediate elements between the coupled elements absent specific contrary language.

The term “configured to” describes hardware, software or a combination of hardware and software that is adapted to, set up, arranged, commanded, altered, modified, built, composed, constructed, designed, or that has any combination of these characteristics to carry out a given function.

“Subject” as used herein is any mammalian or non-mammalian subject. In some embodiments, the subject is a human subject. In some embodiments, the subject is suspected of or diagnosed with cancer. The cancer can be any solid or hematologic malignancy. The cancer can be of any stage and/or grade. Non-limiting examples of cancer include cancers of head & neck, oral cavity, breast, ovary, uterus, gastro-intestinal, colorectal, pancreatic, prostate, brain and central nervous system, skin, thyroid, kidney, bladder, lung, liver, bone and other tissues.

“Tissue” or “tissue sample” as used interchangeably herein, is a biological material obtained from a subject. The tissue can be from any organ or site in the body of the subject. A tissue can be obtained from a subject by any approach known to a person skilled in the art. The tissue can be obtained by surgical resection, surgical biopsy, investigational biopsy or any other therapeutic or diagnostic procedure performed on a subject. In some embodiments, the tissue contains or is suspected to contain tumor cells. The terms tumor cells, cancerous cells, and malignant cells have been used interchangeably. In some embodiments, the tissue is a tumor tissue. In some embodiments, the tissue is obtained from any organ or site in the body of the subject where a cancer has originated or where the cancer has metastasized to. In some embodiments, the tissue may also contain immune cells, stromal cells etc. While the tissue can be in any form (such as frozen or fixed), in preferred embodiments, the tissue is a live, fresh tissue. In some embodiments, the tissue has not been subjected to any tissue fixation techniques known to a person of ordinary skill in the art (such as formalin treatment) or not been stored under any condition or for any duration of time to significantly reduce the number of viable cells.

Tissue fragments are fragments of the tissue sample that have detached from the tissue sample, wherein the fragments are obtained by cutting the tissue in one or more dimensions. In some embodiments, the tissue fragments are obtained by cutting the tissue sample in all three dimensions, such as a first dimension, a second dimension, and a third dimension. In some embodiments, (such as in the case of a biopsy tissue sample) where the tissue sample already has the desired sizes in two dimensions, tissue fragments can be produced by cutting the tissue sample in only one dimension. The tissue fragments can be of various shapes, with non-limiting examples of shapes including cubes, square cuboids, rectangular cuboids, parallelogram prisms and the like. In some embodiments, the tissue fragments are substantially cubical in shape. In some embodiments, the size of each tissue fragment is equal to or less than 1000 μm (such as 1000 μm, 500 μm, 450 μm, 400 μm, 350 μm, 300 μm, 250 μm, 200 μm, 100 μm or 50 μm) in at least one dimension. In some embodiments, the size of each tissue fragment is between 50 μm and 1000 μm in at least one dimension. In some embodiments, the size of each tissue fragment is between 100 μm and 500 μm in at least one dimension. In some embodiments, the size of each tissue fragment is between 150 μm and 350 μm in at least one dimension. In some embodiments, the size of each tissue fragment is between 50 μm and 500 μm (such as 50 μm, 100 μm, 150 μm, 200 μm, 250 μm, 300 μm, 350 μm, 400 μm, 450 μm or 500 μm) in at least two dimensions. In some embodiments, the size of each tissue fragment is between 100 μm and 350 μm in at least two dimensions. In some embodiments, the size of each tissue fragment is between 50 μm and 500 μm in all three dimensions. In some embodiments, the size of each tissue fragment is between 100 μm and 350 μm in all three dimensions. In some embodiments, each tissue fragment is between 300 μm and 350 μm in two dimensions and between 100 μm and 150 μm in a third dimension. In some embodiments, the tissue fragments are uniform in size. As used herein, uniform means substantially uniform, wherein the size of the tissue fragments are within ±30% of one another, in at least one dimension. In some embodiments, the tissue fragments are live tissue fragments, wherein the cutting processes did not substantially reduce the number of viable cells that were present in the tissue sample. In some embodiments, the tissue fragments are live tissue fragments, such that one or more functional assays can be performed on the tissue fragments. A specified size is the desired size of a tissue fragment in one or more dimensions. The specified size can be user-defined or pre-defined depending on tissue type and/or end application. According to one or more embodiments, the tissue cutting system cuts the tissue sample into tissue fragments of a specified size. The size of the tissue fragments is specified in one or more dimensions. In some embodiments, the tissue cutting system cuts the tissue into tissue fragments as per sizes specified in all three dimensions. As used herein, a tissue fragment of a specified size does not necessarily imply that the tissue fragment has the same size in all dimensions. For example, the tissue fragment of a specified size can have the same size in all three dimensions (such as 300 μm×300 μm×300 μm), it can have the same size in two dimensions and a different size in the third dimension (such as 300 μm×300 μm×100 μm), or it can have different sizes in all three dimensions. Tissue fragments that are cut in sizes greater than or less than the specified size (such as in one, two or all three dimensions), depending on the end application, are unwanted tissue fragments. In some embodiments, tissue fragments within ±50% of the specified size (in one or more dimensions) can still be usable or are desired tissue fragments. For example, if the specified size is 300 μm×300 μm×300 μm, tissue fragments with a size of 450 μm in one or more dimensions might still be within the range of specified size (hence desired tissue fragments), however, tissue fragments with size exceeding 450 μm in one or more dimensions might be outside the range of the specified size and hence are unwanted tissue fragments. The size that is acceptable within the range of specified size may be user defined based on the application.

A cutting plane is a plane at which the tissue sample is cut. A cutting event is the process of cutting the tissue at one cutting plane. In some embodiments, the dimensions at which a tissue is cut (that is, the first dimension, the second dimension and the third dimension) are mutually perpendicular to one another. “Successive cutting planes” as used herein refers to a plurality of substantially parallel cutting planes in one dimension.

A cutting process is the process of cutting the tissue sample at a plurality of cutting planes in at least one dimension. In some embodiments, a cutting process comprises multiple cutting events. A first cutting process is the process of cutting the tissue at multiple cutting planes in a first dimension, a second cutting process is the process of cutting the tissue at multiple cutting planes in a second dimension, and a third cutting process is the process of cutting the tissue at multiple cutting planes in a third dimension. In some embodiments, the first, the second, and the third cutting processes are sequential. In some embodiments, the first and the second cutting processes are scoring processes, where the tissue sample is cut at successive planes in the first and the second dimensions respectively, but tissue fragments (i.e., fragments that detach from the tissue sample) are not produced. As to be understood, a “scoring cut” is a cut that cuts a tissue sample but doesn't produce tissue fragments that detach from the tissue sample. As to be understood by a person skilled in the art, a scoring cut is a shallow cut that does not penetrate the entire depth of the tissue sample to produce a fragment that detaches from the tissue sample. A “scored tissue sample” is a tissue sample or a portion thereof that has been cut by scoring cuts in the first and the second dimensions, but tissue fragments (i.e., fragments that detach from the tissue sample) are not produced. In some embodiments, tissue fragments are produced only after the tissue sample is cut in all three dimensions. A “slicing cut” is a cut that produces tissue fragments which detach from the tissue sample. In some embodiments, the cut in the third dimension by the second cutting component (after scoring cuts in the first and the second dimension) is a slicing cut. In some embodiments, especially for biopsy samples (where the tissue sample already has the desired sizes in two dimensions), the cuts in a single dimension produces tissue fragments that detach from the tissue sample and hence is a slicing cut.

A cutting cycle is the sequential or concurrent occurrence of a first cutting process, a second cutting process and a third cutting process to produce tissue fragments. In some embodiments, a cutting cycle is the sequential occurrence of a first cutting process, followed by a second cutting process and finally followed by a third cutting process, to produce tissue fragments. In some embodiments, one cutting cycle cuts only a portion of the tissue sample into tissue fragments. In some embodiments, multiple cutting cycles are required to cut the entire tissue sample into tissue fragments.

A cutting component is any object configured to cut a tissue precisely. The cutting component is configured to cut the tissue precisely at cutting plane in a given dimension with minimal damage to the tissue at the cutting plane or at adjoining regions thereof. According to some embodiments, a cutting plane is a plane at which a cutting component cuts the tissue. Non-limiting examples of cutting components include blades, wires, or scalpels. In some embodiments, the cutting component is a blade.

As used herein, the term “processor” (e.g., a microprocessor, a microcontroller, a controller, a processing unit, or other suitable programmable device) can include, among other things, a control unit, an arithmetic logic unit (“ALC”), and a plurality of registers, and can be implemented using a known computer architecture (e.g., a modified Harvard architecture, a von Neumann architecture, etc.). In some embodiments the processor is a microprocessor that can be configured to communicate in a stand-alone and/or a distributed environment, and can be configured to communicate via wired or wireless communications with other processors, where such one or more processor can be configured to operate on one or more processor-controlled devices that can be similar or different devices.

As used herein, the term “memory” is any memory storage and is a non-transitory computer readable medium. The memory can include, for example, a program storage area and the data storage area. The program storage area and the data storage area can include combinations of different types of memory, such as a ROM, a RAM (e.g., DRAM, SDRAM, etc.), EEPROM, flash memory, a hard disk, a SD card, or other suitable magnetic, optical, physical, or electronic memory devices. The processor can be connected to the memory and execute software instructions that are capable of being stored in a RAM of the memory (e.g., during execution), a ROM of the memory (e.g., on a generally permanent bases), or another non-transitory computer readable medium such as another memory or a disc. In some embodiments, the memory includes one or more processor-readable and accessible memory elements and/or components that can be internal to the processor-controlled device, external to the processor-controlled device, and can be accessed via a wired or wireless network. Software included in the implementation of the methods disclosed herein can be stored in the memory. The software includes, for example, firmware, one or more applications, program data, filters, rules, one or more program modules, and other executable instructions. For example, the processor can be configured to retrieve from the memory and execute, among other things, instructions related to the processes and methods described herein.

As used herein, the term “network” generally refers to any suitable electronic network including, but not limited to, a wide area network (“WAN”) (e.g., a TCP/IP based network), a local area network (“LAN”), a neighborhood area network (“NAN”), a home area network (“HAN”), or personal area network (“PAN”) employing any of a variety of communications protocols, such as Wi-Fi, Bluetooth, ZigBee, etc. In some embodiments, the network is a cellular network, such as, for example, a Global System for Mobile Communications (“GSM”) network, a General Packet Radio Service (“GPRS”) network, an Evolution-Data Optimized (“EV-DO”) network, an Enhanced Data Rates for GSM Evolution (“EDGE”) network, a 3GSM network, a 4GSM network, a 5G New Radio, a Digital Enhanced Cordless Telecommunications (“DECT”) network, a digital AMPS (“IS-136/TDMA”) network, or an Integrated Digital Enhanced Network (“iDEN”) network, etc. In some embodiments, systems comprise a computer and/or data storage provided virtually (e.g., as a cloud computing resource). In particular embodiments, the technology comprises use of cloud computing to provide a virtual computer system that comprises the components and/or performs the functions of a computer as described herein. Thus, in some embodiments, cloud computing provides infrastructure, applications, and software as described herein through a network and/or over the internet. In some embodiments, computing resources (e.g., data analysis, calculation, data storage, application programs, file storage, etc.) are remotely provided over a network (e.g., the internet).

The present disclosure related to a tissue cutting system similar to that disclosed in U.S. patent application Ser. No. 17/941,289, filed Sep. 9, 2022, which is incorporated herein by reference in its entirety.

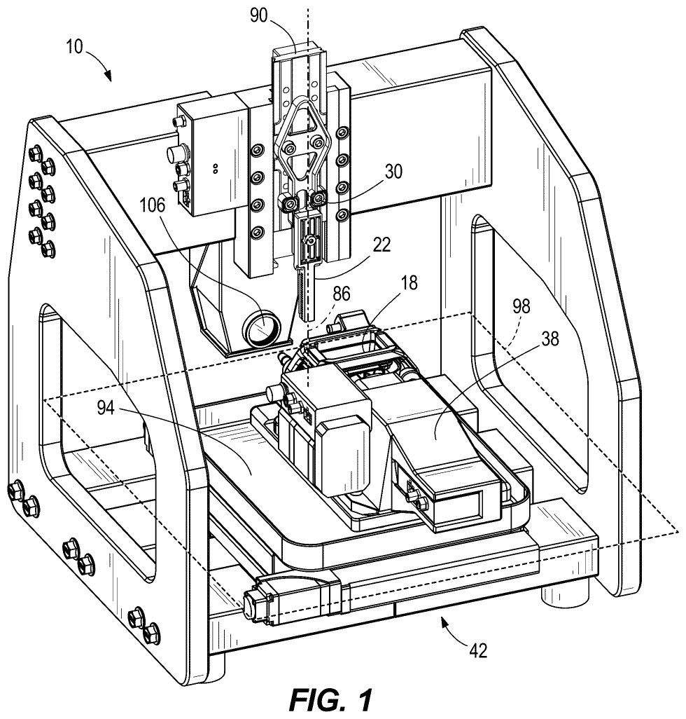

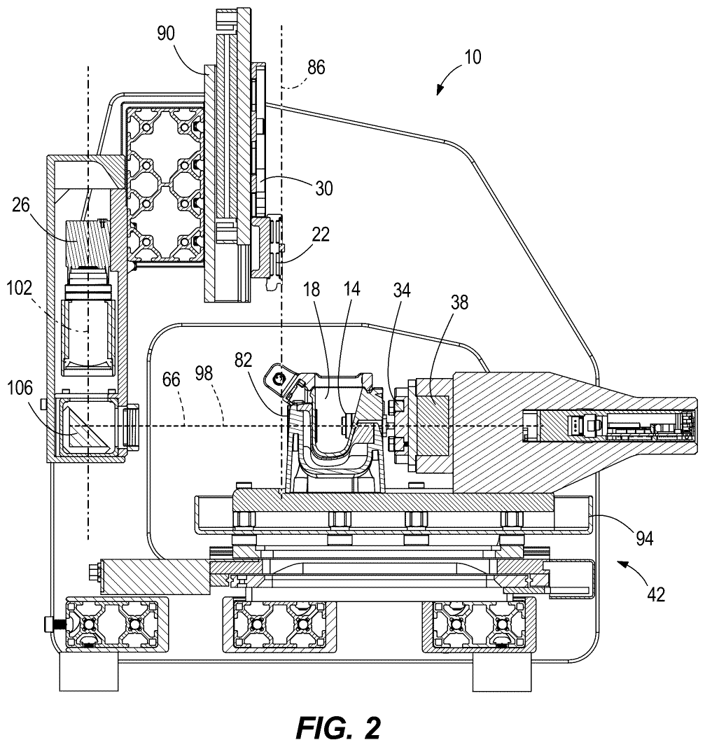

With reference to FIGS. 1 and 2, a tissue cutting system 10 includes a tissue holder 14, a reservoir 18, at least one cutting component 22 (e.g., a blade), and a camera 26. In the illustrated embodiment, the tissue cutting system 10 also includes a blade mount 30, a rotating device 34, a drive assembly 38, and a translation assembly 42.

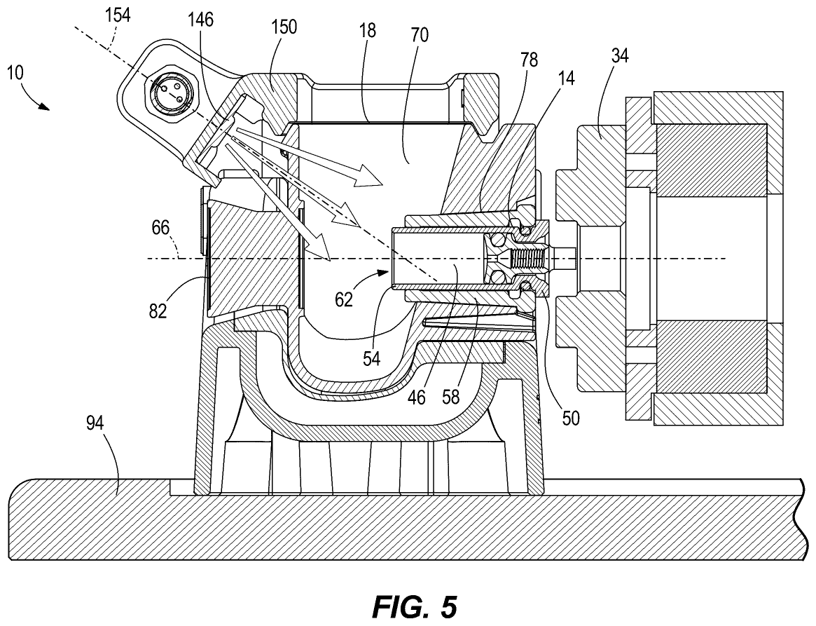

With reference to FIGS. 2 and 5, the tissue holder 14 is configured to hold a tissue sample (e.g., a live tissue sample, a biopsy, an excision, etc.) within a hollow cavity 46. The tissue holder 14 is configured to support the tissue sample. The tissue holder 14 comprises a proximal end 50, a distal end 54, a sidewall 58, the hollow cavity 46 defined by the sidewall 58, and at least one opening 62 at the distal end 54. The opening 62 is configured to expose a portion of the tissue sample. In the illustrated embodiment, a longitudinal axis 66 of the tissue holder 14 is an axis extending between the proximal end 50 and the distal end 54. In the illustrated embodiment, the tissue holder 14 has a circular cross-sectional shape. In other embodiments, the tissue holder has a cross-sectional shape that is elliptical, square, rectangular and the like. In some embodiments, the tissue holder 14 is configured to stably hold the tissue sample within the hollow cavity during the cutting processes. As detailed further herein, the camera 26 is configured to image the opening 62 of the tissue holder 14 and the portion of the tissue sample that is exposed at the opening 62.

With reference to FIGS. 1 and 2, the drive assembly 38 is coupled to the tissue sample and is configured to move the tissue sample relative to the tissue holder 14 along the longitudinal axis 66. The rotating device 34 is coupled to the tissue holder 14 and is configured to rotate the tissue holder 14 about the longitudinal axis 66. In other words, the rotating device 34 rotates the tissue holder 14 and tissue sample between various orientations relative to the cutting component 22.

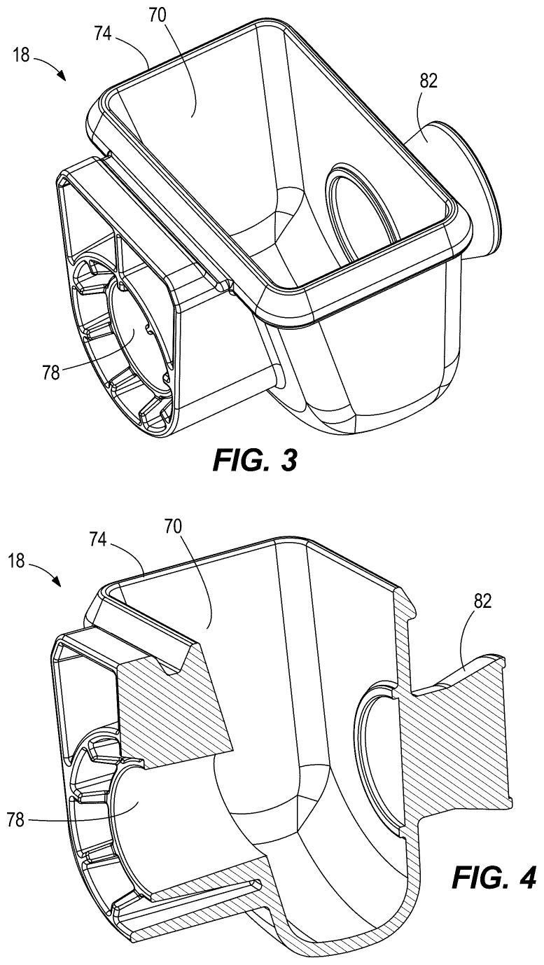

With reference to FIGS. 3 and 4, the reservoir 18 includes an internal space 70 (e.g., a cavity, a bowl, a bucket) and a fluid material is positioned within the internal space 70. The reservoir 18 is configured to collect the tissue fragments within the internal space 70. In the illustrated embodiment, the internal space 70 is at least partially defined by a sidewall 74. The reservoir 70 includes an aperture 78 formed in the sidewall 74 that is configured to at least partially receive the tissue holder 14 (e.g., FIG. 5). In the illustrated embodiment, at least a portion of the tissue holder 14 protrudes into the internal space 70 of the reservoir 18 and is submerged within the fluid material.

With continued reference to FIGS. 3 and 4, the reservoir 18 further includes an optical window 82 (e.g., optical port) in the sidewall 74 positioned opposite the aperture 78. In other words, the optical window 82 is positioned opposite the tissue holder 14 when the tissue holder 14 is inserted through the aperture 78. The window 82 enables visualization by the camera 26 of the tissue holder 14 and the tissue sample submerged in the fluid material in the reservoir 18. In some embodiments, the window 82 is integrally formed with the sidewall 74 and is the same material as the sidewall 74. Integrally forming the window 82 with the sidewall 74 eliminates the risk of a leak occurring at an optical window. Advantageously, the window 82 extends a distance from the sidewall 74 and is configured to prevent fogging during temperature control (e.g., chilling) of the reservoir 18.

With reference to FIGS. 1 and 2, the cutting component 22 is configured to cut the tissue sample contained within the tissue holder 14. In some embodiments, the cutting component 22 is configured to cut a tissue sample contained within the tissue holder 14 by scoring cuts in a first dimension and in a second dimension respectively, to produce a scored tissue sample. In particular, the cutting component 22 is configured to protrude into the cavity 46 (FIG. 5) and cut the tissue sample while the tissue sample is contained within the cavity 46. In some embodiments, the tissue cutting system 10 includes more than one cutting component. In some embodiments, a second cutting component is configured to cut the scored tissue sample in a third dimension to produce tissue fragments.

With reference to FIG. 2, in the illustrated embodiment, the cutting component 22 is positioned on the mount 30, which is translated along an axis 86 by an oscillator 90 (e.g., actuator, voice coil actuator, etc.). The oscillator 90 is configured to move (e.g., oscillate) the mount 30 and the cutting component 22 along the axis 86. In some embodiments, the oscillator 90 is configured to control a frequency and an amplitude of movement for the cutting component 22.

With reference to FIGS. 1 and 2, the translation assembly 42 is coupled to the tissue holder 14 and the reservoir 18. The translation assembly 42 is configured to move the tissue holder 14 (containing the tissue sample) and the reservoir 18 with respect to the cutting component 22 and/or the camera 26. In the illustrated embodiment, the translation assembly 42 includes a translation stage 94 (e.g., a platform) and the reservoir 18 and the tissue holder 14 are mounted to the translation stage 94. In some embodiments, the translation stage 94 is configured to move with two degrees of freedom in a translation plane 98. In the illustrated embodiment, the translation plane 98 is horizontal. In other words, the reservoir 18 and the tissue holder 14 are coupled to the translation stage 98 and movable within the translation plane 98 in response to energizing the translation assembly 42. In the illustrated embodiment, the tissue holder 14 is movable between a first position for cutting by the cutting component 22 and a second position for imaging by the camera 26. In other words, the translation assembly 42 moves the tissue holder 14 and the tissue sample between a cutting position and an imaging position. In some embodiments, the cutting position is the same as the imaging position.

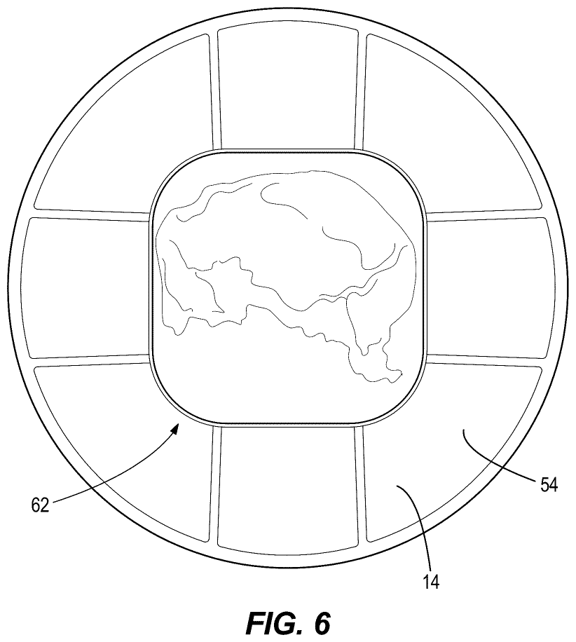

With reference to FIG. 2, the camera 26 defines a camera axis 102. In the illustrated embodiment, the camera axis 102 intersects the translation plane 98. In the illustrated embodiment, the camera axis 102 is vertical and is perpendicular to the horizontal translation plane 98. In the illustrated embodiment, the camera axis 102 is parallel to the axis 86 of the cutting component 22. In the illustrated embodiment, the camera axis 102 is perpendicular to the longitudinal axis 66 of the tissue holder 14. As detailed further herein, the camera 26 is configured to capture images (for example, FIG. 6) that can be analyzed to inform and adjust cutting operations (e.g., target changing, cut settings, end cut operation, etc.) of the cutting component 22. In some embodiments, the camera 26 is a monochromatic camera. In some embodiments, the camera 26 has sensitivity into the ultra-violet (UV), near infrared (NIR), and/or short-wave infrared (SWIR) frequency bandwidth. In some embodiments, the camera 26 is a video camera configured to capture real-time or near real-time video of the tissue sample. The camera provides different imaging performance and/or highlights additional sample properties.

In some embodiments, the tissue cutting system 10 includes a mirror 106. In the illustrated embodiment, the mirror 106 is positioned in the translation plane 98 and is configured to optically direct the field of view of the camera 26 toward the tissue holder 14. In other words, the camera 26 is mounted vertically looking at the mirror 106 that bends the image approximately 90 degrees toward the tissue holder 14. Advantageously, the vertical camera 26 with the mirror 106 results in a smaller form factor that also protects the camera from damage or contamination.

In some embodiments, the tissue cutting system 10 further includes a filter wheel coupled to the camera 26. With the addition of a filter wheel to the camera, florescence imaging in addition to multi-spectral imaging is available to further identify specific properties of a sample as it is processed by the tissue cutting system.

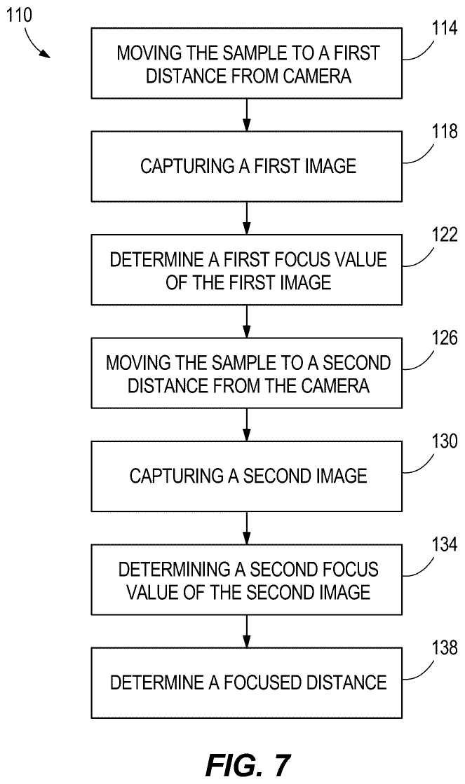

With reference to FIG. 7, a method 110 for determining a focused distance for the tissue sample to be relative to the camera is illustrated. The method 110 includes (STEP 114) moving a sample to a first distance from a camera (e.g., the camera 26); (STEP 118) capturing a first image of the sample with the camera; and (STEP 122) determining a first focus value of the first image. The method further includes (STEP 126) moving the sample to a second distance (different than the first distance) from the camera; (STEP 130) capturing a second image of the sample with the camera; and (STEP 134) determining a second focus value of the second image.

The method 110 includes (STEP 138) determining a focused distance from the camera based on the first focus value and the second focus value. In some embodiments, the method 110 further includes capturing a focused image of the sample with the sample positioned the focused distance from the camera. In some embodiments, the method 110 further includes measuring a surface area of the sample based on the focused image. Advantageously, positioning the sample at the focused distance from the camera allows the focused image to accurately measure the surface area of the sample present. In other words, in order to accurately measure the surface area of the sample present in an image, the sample needs to be in focus. In some embodiments, focus is based on collecting a range of values at different distances and identifying the optimal focus position.

In some embodiments, the method 110 is used as a quality control measure that ensures the sample is loaded properly within the tissue holder. For example, in some embodiments, the method 110 further includes determining the sample is loaded improperly when the first focus value and the second focus value are below a threshold. In other words, if the value returned by the autofocusing method is below a threshold, the method determines the sample is either not present or not properly positioned within the tissue holder.

With reference to FIG. 8, one example of an autofocus workflow 142 is illustrated. The autofocus workflow 142 is used with the tissue cutting system 10 to ensure the face of the sample is completely in focus while identifying a target area, for example. The autofocus workflow 142 advantageously improves consistency and accuracy throughout the cutting process. If the sample is out of focus, distortion is introduced, and a location selected by the user for cutting (detailed further herein) will not be accurately represented by the cutting operation.

With reference to FIG. 5, in some embodiments, the tissue cutting system 10 further includes a light source 146 (e.g., a light emitting diode) configured to illuminate the opening 62 of the tissue holder 14. Advantageously, the light source 146 provides consistent illumination of the tissue sample. In some embodiments, the light source 146 is an array of light emitting diodes. In some embodiments, the fluid material in the reservoir 18 is selected to transmit light uniformly to illuminate a face of the sample.

In some embodiments, the light source 146 is coupled to the reservoir. In the illustrated embodiment of FIG. 5, the light source 146 is coupled to a cover 150 on the reservoir 18. With reference to FIG. 5, the light source 146 defines an illumination axis 154 that intersects the longitudinal axis 66 of the tissue holder 14. In the illustrated embodiment, the light source 146 is integrated into the cover 150 and projects light directly at the face of the sample and not at a user. In some embodiments, the light source 146 is intermittently energized according to when the camera 26 captures an image. In other words, the light source 146 is only energized to emit a light when the camera 26 is capturing an image. Advantageously, the intermittent energization of the light source 146 reduces the potential for heat generated by the light source 146 from affecting the temperature-controlled reservoir 18 and tissue sample.

In some embodiments, the light source 146 is configured to be energized to emit light with different spectral power distributions. For example, the light source 146 is energized to emit a first light with a first spectral power distribution and is energized to emit a second light with a second spectral power distribution. In some embodiments, the light source 146 is configured for multi-spectral illumination. In some embodiments, the dominant amount and frequency or frequencies of light illuminating the sample during imaging is control. In some embodiments, a single RGB (red, green, and blue) image is captured or multiple separate images at select frequencies are taken and combined into a composite image.

In some embodiments, the light source 146 is positioned at least partially within the reservoir. In some embodiments, the light source is positioned outside the reservoir and shining through the wall. In some embodiments, the sidewall of the reservoir includes a light shaping feature (e.g., a Fresnel element).

In some embodiments, the tissue cutting system further includes a light pipe configured to direct a light emitted from the light source 146 to the opening of the tissue holder 14. In some embodiments, the light pipe is formed in the reservoir.

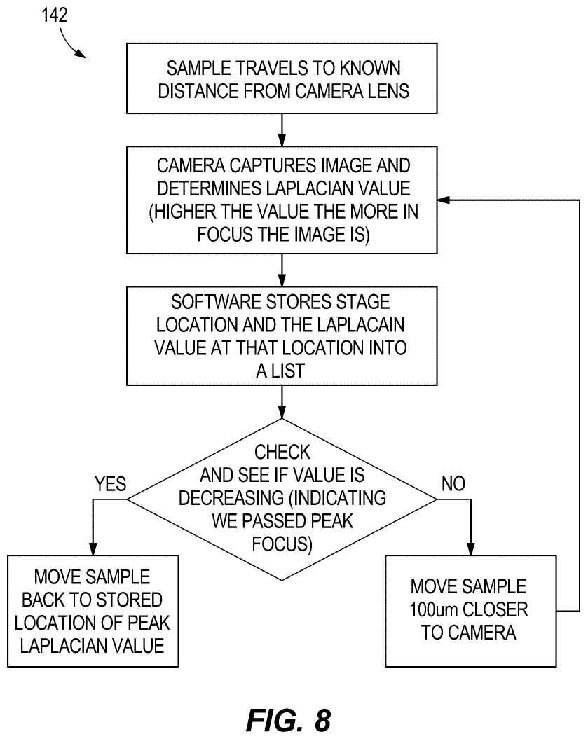

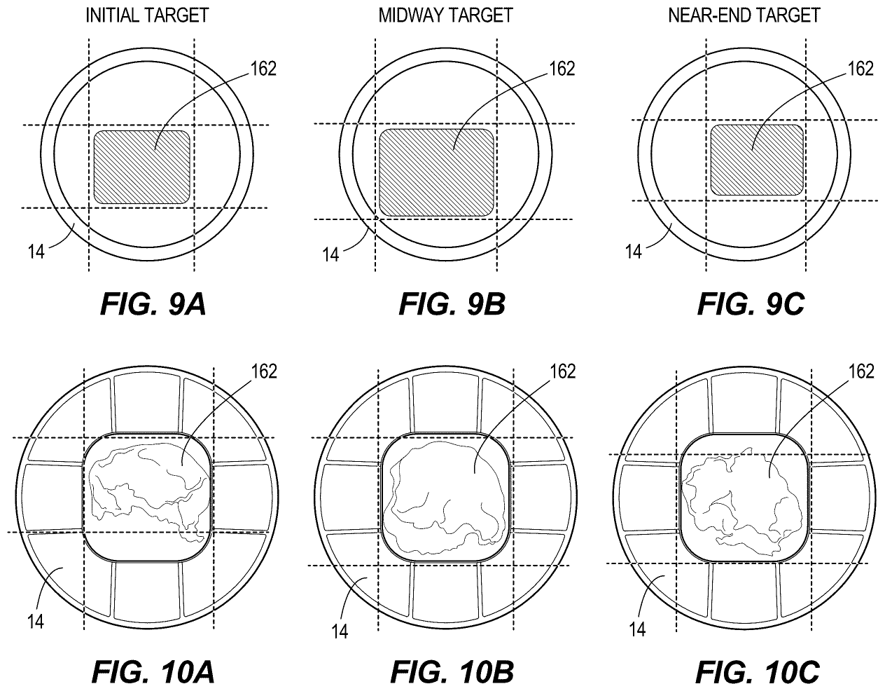

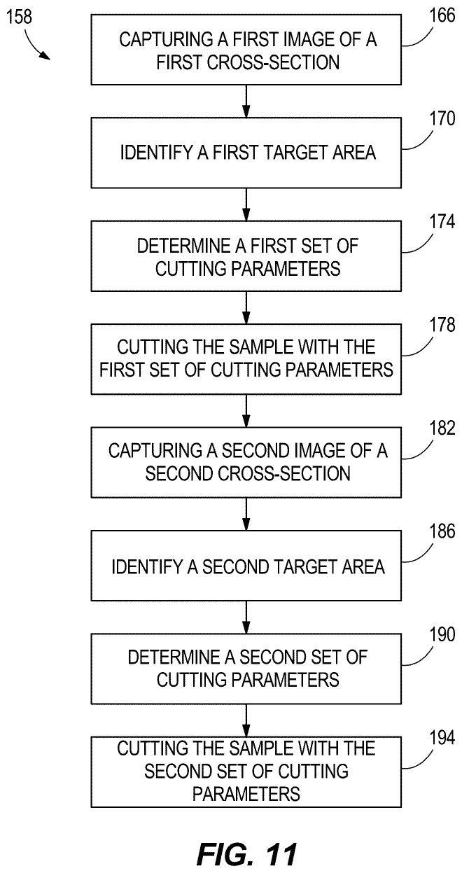

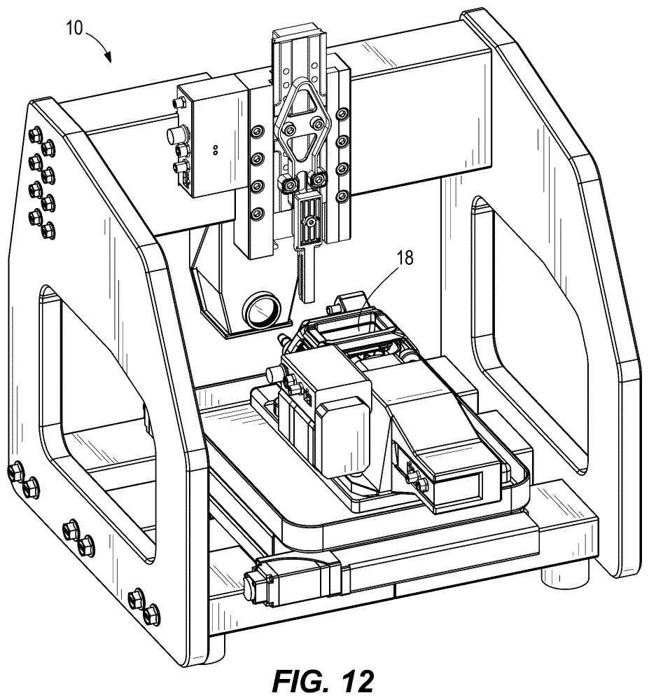

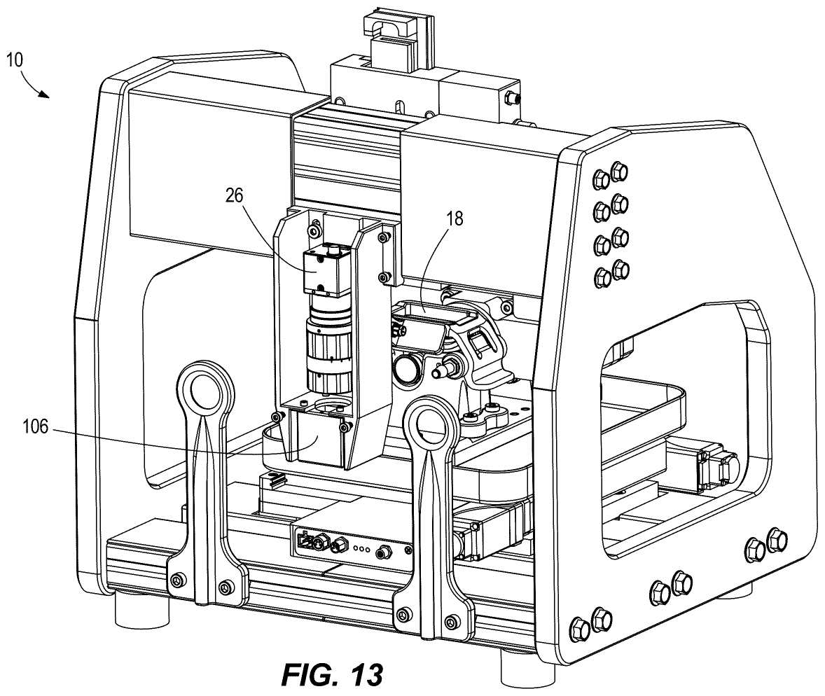

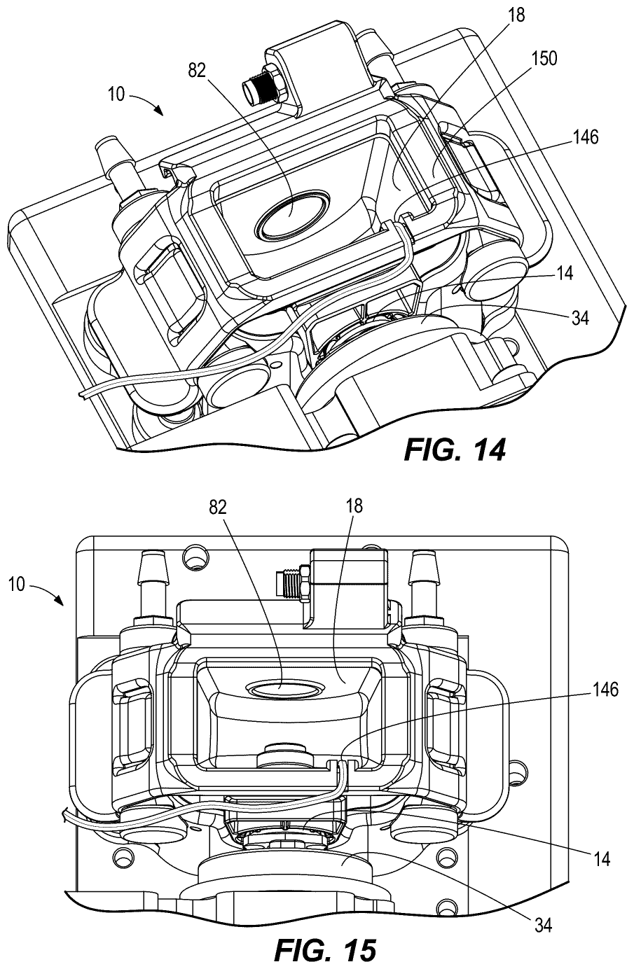

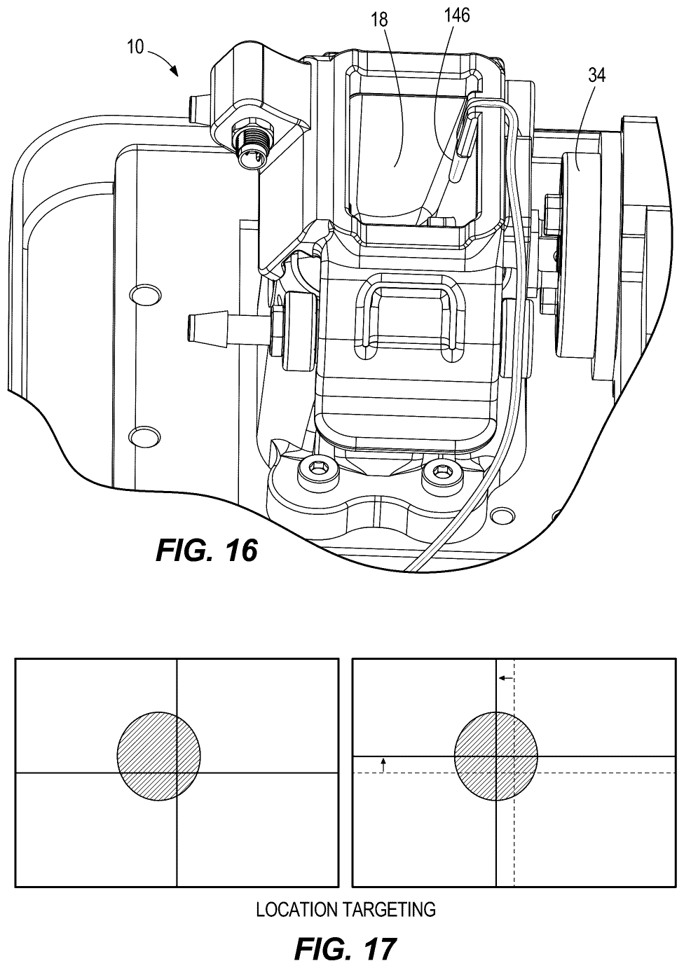

With reference to FIGS. 11, a method 158 of targeting a portion of a sample and adjusting cutting parameters is illustrated. Tumor excisions and biopsies vary in properties such as size, shape, density, and tissue type. Differences in morphology and effects of the mounting process can cause shifts in the relative positioning of the sample along the length of the sample. For example, FIGS. 9A-9C schematically illustrate how a target area 162 can vary at different cross-sections in the tissue sample. FIGS. 10A-10C are images captured by the camera 26 that illustrate how a target area 162 varies at different cross-sections in a tissue sample. FIGS. 9A-10C illustrate how the tissue cutting system can dynamically retarget an area of interest to be cut as the cutting cycle processes through the sample. In other words, the cross-sectional area and the relative location of the target region changes from slice to slice and imaging the tissue sample throughout the cutting cycle improves the efficiency and functionality of the cutting system 10. Without a dynamic retargeting of the sample, for example, part of the sample will be left uncut and/or time is wasted cutting the sample with no region of interest. In some embodiments, the tissue holder is rotated about the longitudinal axis 66 by the rotating device 34 during the process of re-targeting the sample.

With reference to FIGS. 17-19, various types of dynamic targeting (in a first instance) or re-targeting (in second instance) are illustrated. With reference to FIG. 17, a biopsy tissue sample that is round in cross section can be dynamically targeted to move to a center location such that the biopsy tissue sample is cut with no rotation of the sample or the tissue holder about the longitudinal axis 66. The result of the dynamic re-targeting in FIG. 17 is four tissue fragments that are all approximately the same size.

In some cases, the cross-sectional shape of the tissue sample (e.g., a biopsy) is not circular but oblong or another irregular shape. Although the cross section of biopsies are depicted herein as being symmetrical, this is not always the case and sometimes the biopsy is asymmetrical with variability in shape.

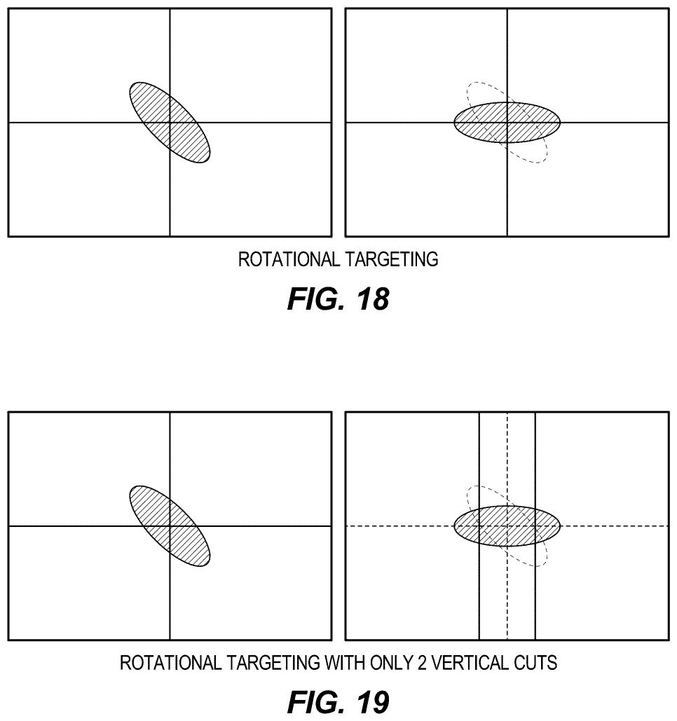

With reference to FIG. 18, when a biopsy tissue sample is not round but more oblong in cross-section, cutting the biopsy tissue sample without rotation of the sample and the tissue holder about the longitudinal axis 66 would yield four tissue fragments of unequal size (Left panel of FIG. 18). The methods disclosed herein include rotating the sample and the tissue holder around the longitudinal axis 66 before cutting occurs in a dynamic rotational targeting (Right panel of FIG. 18). The result of the dynamic re-targeting of FIG. 18 is four tissue fragments that are all approximately the same size. With reference to FIG. 19, a rotational targeting with two vertical cuts, for example, is illustrated. In other words, the oblong biopsy is rotated and then a cut pattern with two vertical lines is utilized to improve tissue fragment similarity. Alternative cutting strategies can be considered and deployed with the tissue cutting system to optimize similarity in the size of tissue fragments.

In some embodiments, the camera of the tissue cutting system captures and assesses a surface of the biopsy cross section and utilizes machine vision to determine an optimal cutting strategy and/or pattern for achieving a desired outcome. In some embodiments, the desired outcome is equivalency in fragment size for any given target dimension and/or shape.

To optimally process an individual sample, it is advantageous to actively track, assess, and target a cutting or scoring pattern to the current cross-section of the sample being cut or scored. The method 158 includes (STEP 166) capturing a first image of a first cross-section of a sample (e.g., FIG. 9A, 10A); (STEP 170) identifying a first target area in the first image; (STEP 174) determining a first set of cutting parameters based on the first target area; and (STEP 178) cutting the sample with the first set of cutting parameters. The method 158 further includes (STEP 182) capturing a second image of a second cross-section of the sample (e.g., FIG. 9B, 10B). As such, the camera 26 images the tissue sample after the cutting component has cut the tissue sample. The method 158 further includes (STEP 186) identifying a second target area in the second image; (STEP 190) determining a second set of cutting parameters based on the second target area; and (STEP 194) cutting the sample with the second set of cutting parameters. In some embodiments, the method 158 further includes determining a target volume based on the first image and the second image.

In some embodiments, the cutting parameters include a scoring pattern. In some embodiments, the scoring pattern is a variable sized scoring pattern (e.g., a scoring pattern with unequal distances between adjacent scores). In some embodiments, cutting the sample creates a plurality of target tissue fragments of a first size (e.g., volume) and a plurality of waste fragments of a second size (e.g., volume). In some embodiments, the second size is larger than the first size. In some embodiments, the waste fragments are comprised of multiple size and shaped fragments Advantageously, waste fragments that are larger can be more easily identified and discarded in a downstream sorting process.

In some embodiments, one or more component of the tissue cutting system are disposable and/or sterilizable. In some embodiments, during operation, the tissue cutting system forms a closed system in order to maintain the sterility of the tissue sample. In some embodiments, the tissue cutting system is an automated or a semi-automated system, wherein the components of the system are configured to perform their designated functions with minimal human intervention.

The sequential events of the first, the second and the third cutting processes constitutes a cutting cycle, such as a first cutting cycle. In some embodiments, after a cutting cycle (such as a first cutting cycle), the tissue holder and the reservoir are moved back to the original position, such as by the translation assembly. In some embodiments, one cutting cycle cuts only a portion of the tissue sample into tissue fragments. In some embodiments, the first, the second and the third cutting processes are repeated for plurality of cutting cycles, wherein substantially the entire tissue sample is cut into tissue fragments. In some embodiments, one or more steps of the method of cutting the tissue sample into tissue fragments are automated or semi-automated, that is the steps are performed with minimal human intervention.

What is claimed is:

1. A tissue cutting system comprising:
a tissue holder configured to hold a tissue sample, the tissue holder includes an opening configured to expose a portion of the tissue sample;
a reservoir, wherein a portion of the tissue holder is positioned within the reservoir;
a cutting component configured to cut the tissue sample contained within the tissue holder; and
a camera configured to image the opening of the tissue holder and the portion of the tissue sample.

2. The tissue cutting system of claim 1, wherein the tissue holder is movable between a first position for cutting by the cutting component and a second position for imaging by the camera.

3. The tissue cutting system of claim 1, further comprising a translation assembly coupled to the tissue holder and configured to move the tissue holder containing the tissue sample with respect to the cutting component, the camera, or a combination thereof.

4. The tissue cutting system of claim 3, wherein the translation assembly includes a translation stage, the reservoir and the tissue holder are mounted to the translation stage.

5. The tissue cutting system of claim 4, wherein the translation stage is configured to move in two degrees of freedom in a translation plane.

6. The tissue cutting system of claim 5, wherein the camera defines a camera axis that intersects the translation plane.

7. The tissue cutting system of claim 5, further including a mirror positioned in the translation plane.

8. The tissue cutting system of claim 1, wherein the camera defines a camera axis that is perpendicular to a longitudinal axis of the tissue holder.

9. The tissue cutting system of claim 1, further comprising a light source configured to illuminate the opening of the tissue holder.

10. The tissue cutting system of claim 9, wherein the light source is coupled to the reservoir or a cover on the reservoir.

11. The tissue cutting system of claim 9, wherein the light source defines an illumination axis that intersects a longitudinal axis of the tissue holder.

12. The tissue cutting system of claim 9, wherein the light source is intermittently energized according to when the camera captures an image of the opening of the tissue holder.

13. The tissue cutting system of claim 9, wherein the light source is energized to emit a first light with a first spectral distribution and is also energized to emit a second light with a second spectral distribution.

14. The tissue cutting system of claim 9, further including a light pipe configured to direct a light emitted from the light source to the opening of the tissue holder.

15. The tissue cutting system of claim 14, wherein the light pipe is formed in the reservoir.

16. The tissue cutting system of claim 9, wherein the light source is positioned at least partially within the reservoir.

17. The tissue cutting system of claim 1, further including a filter wheel coupled to the camera.

18. The tissue cutting system of claim 1, wherein the reservoir is filled with a fluid material, wherein the portion of the tissue holder is submerged within the fluid material.

19. The tissue cutting system of claim 18, wherein the reservoir includes an optical window positioned opposite the tissue holder.

20. The tissue cutting system of claim 1, further comprising a drive assembly configured to move the tissue sample relative to the tissue holder along a longitudinal axis of the tissue holder, and a rotating device coupled to the tissue holder and is configured to rotate the tissue holder about the longitudinal axis.

21. The tissue cutting system of claim 1, wherein the cutting component is a first cutting component configured to cut a tissue sample contained within the tissue holder by scoring cuts in a first dimension and a second dimension, and wherein the tissue cutting system further includes a second cutting component configured to cut the tissue sample in a third dimension to produce tissue fragments.

22. The tissue cutting system of claim 1, wherein the camera images the tissue sample after the cutting component has cut the tissue sample.

23. The tissue cutting system of claim 1, wherein the tissue sample is a live tissue sample.

24. A method comprising:
capturing a first image of a first cross-section of a sample;
identifying a first target area in the first image;
determining a first set of cutting parameters based on the first target area;
cutting the sample with the first set of cutting parameters;
capturing a second image of a second cross-section of the sample;
identifying a second target area in the second image;
determining a second set of cutting parameters based on the second target area; and
cutting the sample with the second set of cutting parameters.

25. The method of claim 24, further comprising determining a target volume based on the first image and the second image.

26. The method of claim 24, wherein the first set of cutting parameters includes a variable sized scoring pattern.

27. The method of claim 24, wherein cutting the sample creates a plurality of target tissue fragments of a first size and a plurality of waste fragments of a second size.

28. The method of claim 27, wherein the second size is larger than the first size.

29. A method comprising:
moving a sample to a first distance from a camera;
capturing a first image of the sample with the camera;
determining a first focus value of the first image;
moving the sample to a second distance from the camera;
capturing a second image of the sample with the camera;
determining a second focus value of the second image; and
determining a focused distance from the camera based on the first focus value and the second focus value.

30. The method of claim 29, further comprising capturing a focused image of the sample with the camera with the sample positioned the focused distance from the camera.

31. The method of claim 30, further including measuring a surface area of the sample based on the focused image.

32. The method of claim 29, further including determining the sample is loaded improperly when the first focus value and the second focus value are below a threshold.
